(19) United States
(12) Patent Application Publication
(10) Pub. No.: US 2002/0014238 A1
(43) Pub. Date: Feb. 7, 2002

(54) METHOD AND APPARATUS FOR REMOVING COLLECTED SECRETIONS FROM CUFFED VENTILATION TUBE IN A PATIENT'S TRACHEA

(76) Inventor: ROBERT F. KOTMEL, BURLINGAME, CA (US)

Correspondence Address: JAMES M HESLIN, TOWNSEND AND TOWNSEND AND CREW, TWO EMBARCADERO CENTER, 8TH FLOOR, SAN FRANCISCO, CA, 941113834

(21) Appl. No.: 09/249,040

(22) Filed: Feb. 12, 1999

(51) Int. Cl. A62B 7/00; A61M 16/00; A62B 9/06

(52) U.S. Cl. 128/204.18

(57) ABSTRACT

A ventilation device comprises a ventilation tube having an inflatable occlusion cuff near its distal end. A deployable isolation barrier, such as secondary isolation cuff, is spaced proximally from the inflatable occlusion cuff on the ventilation tube. Deployment of the isolation barrier while the ventilation tube is in use creates an isolated region within a patient's trachea for capturing accumulated secretions and facilitating removal of such secretions. Usually, the isolated region will be irrigated to disperse the secretions and aspirated to remove the irrigant and dispersed secretions.

### BACKGROUND OF THE INVENTION

1. Field of the Invention

The present invention relates generally to medical devices, methods, and kits. More particularly, the present invention relates to patient ventilation tubes having improved designs which facilitate removal of secretions which collect over an occlusion cuff on the ventilation tube.

Patients are intubated with endotracheal and other ventilation tubes for a variety of reasons. For example, patients receiving anesthetics may be ventilated for relatively short periods of time. Patients suffering from respiratory failure, in contrast, may be intubated and ventilated for days, weeks, or even longer while in intensive care. Ventilation tubes which enter the trachea through a patient's mouth or nose are generally referred to as endotracheal tubes. Ventilation tubes which are inserted through an incision in the neck are usually referred to as tracheostomy tubes. In both cases, the devices will include a ventilation tube having a central lumen to provide for ventilation of the patient. Additionally, an inflatable cuff is provided over the exterior surface of the ventilation tube to permit occlusion of the annular space which surrounds the ventilation tube when placed inside the trachea. It is desirable to occlude this annular space in order to facilitate pressurization and ventilation as well as to prevent the intrusion food or other of substances into the lungs.

The presence of the inflatable occlusion cuff over the ventilation tube, however, leads to the accumulation of secretions from the oral-nasal pharynx. These secretions, referred to as subglottic secretions, collect over the upper surface of the inflatable cuff over time. The secretions can migrate downward past the inflated occlusion cuff and will sometimes inoculate the bronchus with bacteria, potentially causing the nosocomial pneumonia. For these reasons, it is desirable to remove the liquid secretions which accumulate on top of the occlusion cuffs on various ventilation tubes.

Conventional method for removing such secretions rely on use of a separate aspiration tube to periodically collect the secretions. A nurse or other trained individual will blindly introduce an aspiration tubedown the patient's throat and attempt to collect as much as the secretions as possible. Alternatively, a separate aspiration tube may be introduced through the main lumen of the endotracheal tube and just past its distal end. Suction is applied through the aspiration tube as the endotracheal cuff is let down, and accumulations which flow downward are aspirated. Such approaches, however, are rarely effective in removing all of the secretions, and the patient remains at significant risk of infection.

As an improvement over the use of a separate aspiration tube, a variety of designs for endotracheal and other ventilation tubes having intrinsic aspiration lumens have been proposed. Of particular interest to the present invention, endotracheal tubes having separate irrigation and aspiration tubes on their exterior surfaces have been proposed for cleaning the upper surface of the endotracheal cuffs. While such designs can provide a reasonably thorough cleaning under certain circumstances, there is still a significant risk that secretions will remain in pockets or other areas on the tube surface which are difficult to reach and clean.

For these reasons, it would be desirable to provide improved devices and methods for the in situ cleaning of endotracheal cuffs on endotracheal and other ventilation tubes during use. In particular, it would be desirable to provide methods and devices which can achieve a very thorough removal of secretions from the upper surfaces of such endotracheal tube or tracheostomy cuffs. Preferably, the devices and methods could be achieved with only minor modification of conventional endotracheal or other ventilation tube designs. Still further desirable, such devices and methods should be useful in hospitals and other settings, particularly being useful in intensive care settings where they can be used without significant additional attention from the hospital staff. For example, it would be desirable if such devices and methods were adaptable for automatic implementation so that they could be implemented without constant staff attention.

2. Description of the Background Art

A number of patents disclose endotracheal tubes, having lumens or secondary tubes for aspirating/irrigating secretion from over an endotracheal cuff. See, e.g., U.S. Pat. Nos. 5,582,167; 5,520,175; 5,501,215; 5,311,864; 5,201,310; 5,143,062; 5,067,497; 4,840,173; 4,637,389; and 4,305,392. U.S. Pat. No. 5,582,167, describes a system which combines automatic control, the use of antibiotic irrigation solutions, and a coaxial suction tube. Endotracheal tubes having dual cuffs are described in U.S. Pat. Nos. 5,033,466; 4,979,505; 4,976,261; and 4,091,816. Apparatus for cleaning the interior of an endotracheal tube are described in U.S. Pat. Nos. 5,709,691 and 5,687,714. An endotracheal tube impregnated with silver ions to inhibit infection is described in U.S. Pat. No. 5,725,510. A dual balloon infusion catheter is described in U.S. Pat. No. 5,460,610. A dialysis system comprising coaxial ballooned catheter for flushing the large intestine is described in U.S. Pat. No. 5,620,604.

### SUMMARY OF THE INVENTION

The present invention provides improved ventilation devices, ventilation device kits, and methods for ventilating patients. The improvements specifically provide for the removal of secretions which can accumulate over the inflatable occlusion cuffs of such devices, as discussed above. In particular, the present invention provides methods and apparatus which isolate a region over the cuff to facilitate and enhance the removal of the accumulated secretions. The secretions will be located within the isolated region, and the region will thereafter be aspirated by applying a negative pressure to draw the secretions away from the occlusion cuff and remove them from the patient. Usually, the isolated region will be irrigated prior to aspiration in order to mobilize the pooled secretions and facilitate their removal. Optionally, the irrigant may be introduced continuously while the region is aspirated in order to wash the region above the occlusion cuff. Such methods can achieve very high levels of cleaning in order to significantly reduce the risk of the secretions migrating past the occlusion cuff and causing infections of the respiratory tract.

In the exemplary embodiments, isolation is achieved with a secondary inflatable cuff which is spaced above the primary (occlusion) cuff, typically by distance in the range from 0.25 cm to 10 cm, usually from 1 cm to 2 cm. Such isolated regions will usually have a volume in the range from 0.05 cc to 80 cc, typically from 3 cc to 10 cc. The primary occlusion cuff will generally remain inflated for extended periods of time (consistent with normal patient ventilation practices), and the secondary cuff will periodically be inflated, usually just prior to removal of the secretions. After inflating the secondary cuff, the region will be irrigated and aspirated, either sequentially or simultaneously, according to methods described in more detail below. The isolated region will thus be washed until the secretions have been removed to below a desired level, and the secondary cuff will then be deflated. The secondary cuff will usually remain deflated at all times other than when the secretions are being removed. Maintaining the secondary cuff in its deflated condition (or other isolation barrier in a non-deployed condition) is advantageous as it permits the secretions to accumulate over the primary occlusion cuff. Presence of the secretions over the primary occlusion cuff is necessary, of course, to permit their removal using the device and methods of present invention. It is permissible that a small amount of secretions may accumulate over the secondary inflatable cuff, but it is preferable to reduce that amount so that since those secretions will generally pass to the primary occlusion cuff when the secondary cuff is deflated.

Ventilation devices according to the present invention comprise a ventilation tube having a proximal end and a distal end. An inflatable cuff is disposed on the ventilation tube near its distal end, and an isolation barrier, typically an inflatable secondary cuff, is located just proximally of the primary inflatable cuff. One or more aspiration lumens are provided on the ventilation tube with a port between the inflatable cuff and the isolation barrier and a port near the proximal end of the ventilation tube. The aspiration lumen permits aspiration of the isolated region formed between the inflated cuff and the isolation barrier to remove the accumulated secretions, as discussed above. The aspiration lumen may also be used to introduce irrigant to facilitate washing of the isolated region, but often one or more additional irrigation lumen(s) will be provided on or in the ventilation tube so that irrigation and aspiration of the isolated region is facilitated. Alternatively, the aspiration lumen may be provided with two separate external ports to permit separate connection to both aspiration source and an irrigation source. In all cases, the aspiration/irrigation lumens may be formed as channels or luminal voids within the wall of the ventilation tube or may be alternatively formed as separate tubes located externally to the ventilation tube or internally within the primary ventilation lumen of the ventilation tube.

Both the primary occlusion cuff and the secondary isolation cuff will usually be inflated in order to cause radial expansion within the patient's trachea. In some instances, however, either of both the cuffs may be radially expanded in their “shelf” or normal condition and be radially collapsed by drawing a negative deflection pressure on the cuff. For convenience, both types of cuffs will be referred to hereinafter as “inflatable” although it might be more technically correct to refer to the second type as “deflatable.”

The ventilation devices of the present invention may be incorporated into systems which facilitate deployment and management of the ventilation device. Such systems typically comprise a source of cuff inflation medium, a source of irrigant solution, a vacuum disposal source, and a controller. The controller may be a digital controller, a computer, an elecromechanical programmable controller, or any other control system of a type capable of operating valves, solenoids, timers, making pressure measurements, and the like. The controller of the present invention will selectively control the inflation of at least the secondary inflatable cuff, usually controlling inflation of both of the primary occlusion cuff and the secondary inflatable cuff. The controller will also control delivery of irrigation fluid to the isolated region between the first and second cuffs as well as controlling the aspiration of the irrigant from the isolated region to the vacuum disposal source. Before delivering the irrigation fluid, the controller will preferably verify that the main cuff is properly inflated to assure isolation of the lung. The irrigation solution may simply be saline, but can also incorporate anti-bacterial agents, anti-fungal agents, anti-viral, mucolytic agents, and combinations thereof. The irrigation medium source can be simply a fluid bag, or could further comprise active pumping means to deliver the fluid a desired pressure, typically in the range from 1 mmHg to 20 mmHg, typically from 10 mmHg to 20 mmHg. The inflation medium source may be pressurized air or other gas available at the treatment facility, or could comprise a dedicated pump and pressurization system. The pressurized medium, typically a gas, will be available at a pressure from 10 mmHg to 40 nmHg, typically from 15 mmHg to 20 mmHg. This source will be suitable for both the primary occlusion cuff and the secondary isolation cuff. Controller may be manual, i.e. requiring manual initiation of a wash and aspiration cycle, but will more typically be operated with a timer on a cyclic basis. Typical duty cycle times (i.e., times between aspirations) will be in the range from 10 seconds to 10 minutes, usually from 60 seconds to 5 minutes. A variety of other control schemes would also be feasible. For example, it would be possible to maintain the aspiration of the region continuously (at least while the main isolation cuff remains inflated) and to periodically infuse the irrigant solution. With continuous aspiration, it would be possible to reduce the frequency of irrigant introduction, possibly to as little as once per day or once every several hours. It would still be possible, of course, to introduce the irrigant on the more frequent duty cycle set forth above.

Methods according to the present invention for removing accumulated secretions from over ventilation tube cuff present in a patient's trachea comprise deploying an isolation barrier over the cuff to isolate a region in the trachea between the barrier and the cuff. The accumulated secretions may then be aspirated from within the isolated region and removed from the region, typically through a lumen and the ventilation device. Usually, isolation barrier will be opened during those periods when the accumulations are not being aspirated, and more usually the cycle of deploying the isolation barrier, aspirating the accumulated secretions, and opening the isolation barrier will be repeated periodically with the duty cycles set forth above. Usually, the isolation barrier comprises a second inflatable cuff, and preferably the isolated region is irrigated with a suitable irrigation solution prior to or simultaneously with the aspiration of the accumulated secretions.

### BRIEF DESCRIPTION OF THE DRAWINGS

FIG. 1 illustrates an exemplary embodiment of a ventilation device constructed in accordance with the principles of the present invention.

FIG. 1A illustrates an alternative cuff configuration for the ventilator device of FIG. 1.

FIGS. 2A and 2B are cross-sectional views taken along lines 2A and 2B of FIG. 1, respectively.

FIGS. 3-6 illustrate alternative cross-sectional designs of the ventilation device of FIG. 1.

FIG. 7 illustrates a ventilation system incorporating the ventilation device of FIG. 1 with a control system.

FIGS. 8A-8C illustrate a method for removing accumulated secretions performed in accordance with the principles of the present invention.

### DESCRIPTION OF THE SPECIFIC EMBODIMENTS

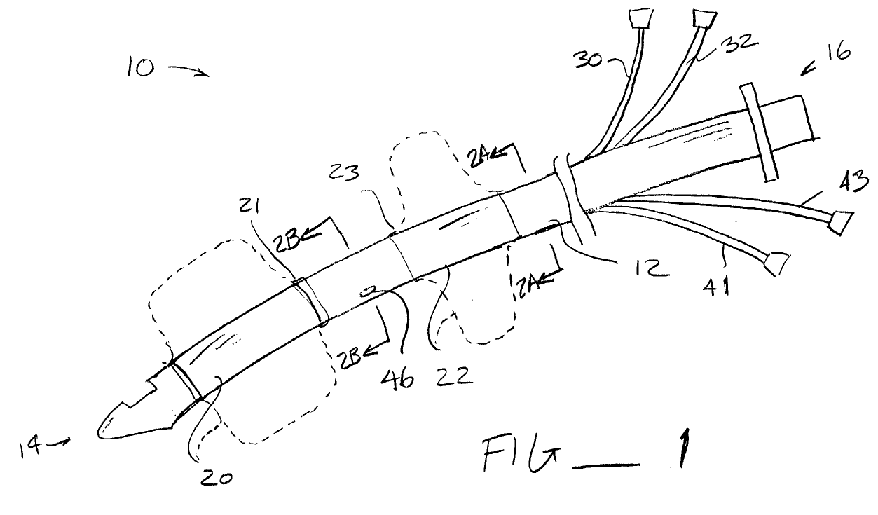

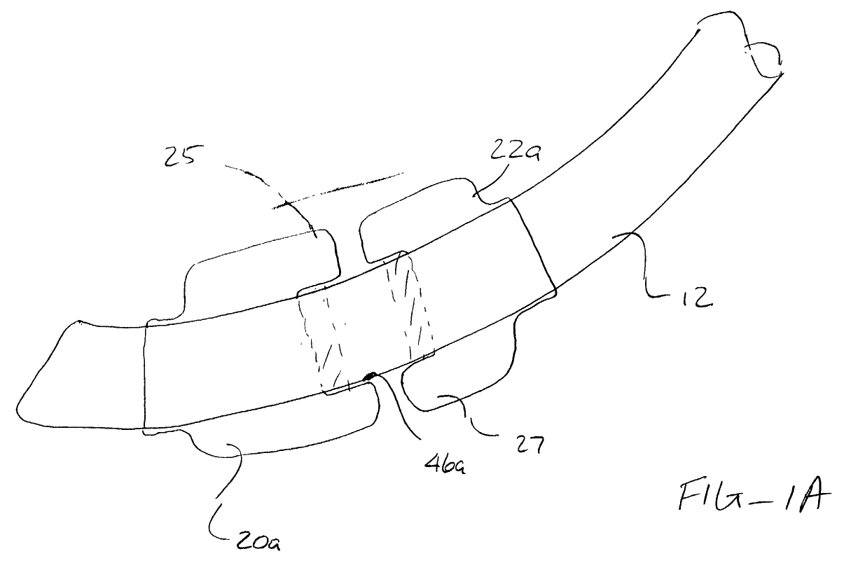

Referring now to FIGS. 1, 2A, and 2B, A ventilation device 10 constructed in accordance with the principles of the present invention will be described. Ventilation device 10 comprises ventilation tube 12 having a distal end 14 and a proximal end 16. The distal end 14 is adapted to be positioned with the throat or trachea of a patient to deliver air in order to mechanically ventilate the patient (and optionally deliver anesthetics or other bioactive agents) in a conventional manner. Proximal end 16 will be adapted to be connected to a conventional mechanical ventilation system (not illustrated). The ventilation tube 12 will typically have a length in the range from about 15 cm to 35 cm, an outside range diameter from 4 mm to 12 mm (although in certain embodiments the ventilation tube of the present invention will have a non-circular cross-section), and a ventilation lumen 18 having a diameter in the range from 2.5 mm to 10 mm. The ventilation device 10 further comprises a primary occlusion cuff 20 which may be inflated from a collapsed or low profile configuration (shown in solid line) to an expanded or inflated configuration (shown in broken line in FIG. 1). The construction, dimensions, and other aspects of the primary occlusion cuff 20 will generally be conventional. The ventilation device 10 will further comprise a secondary or isolation cuff 22 which is spaced proximally of the primary cuff 20. The secondary or isolation cuff 22 will also be inflatable and may have dimensions generally the same of those of the primary cuff 20. As illustrated, however, the length of the secondary cuff 22 is somewhat shorter of that of the primary cuff 20. While an inflatable secondary cuff 22 is illustrated, it will be appreciated that the present invention could also employ other deployable isolation barriers, such as collars, rings, flanges, or other barriers that can be opened radially outward to seal against the inner surface of the trachea after the ventilation device has been deployed in a patient's trachea.

A primary inflation tube 30 is fluidly coupled to the primary occlusion cuff 20 to permit inflation of the cuff when the ventilation device 10 is deployed in a patient's trachea. A secondary inflation tube 32 is fluidly coupled to the secondary inflation cuff 22 to likewise permit of inflation of the secondary cuff when the device is deployed. Inflation tubes 30 and 32 may comprise separate tubes attached to the exterior of the ventilation tube 12, as generally shown in FIGS. 2A and 2B, but could also comprise in whole or in part lumens formed in the wall of the ventilation tube 12, as described hereinafter.

Of particular interest to the present invention, an irrigation lumen 40 is provided in the wall of the ventilation tube 12 and terminates in an external connector 41 at the proximal end of the device. Similarly, an aspiration lumen 42 is located in the wall of the device and terminates in an external connector 43 at the proximal end of the device. The irrigation lumen terminates at a port 44 disposed between the primary occlusion cuff 20 and secondary isolation cuff 22, while the aspiration lumen 42 terminates in a port 46, also located between the two cuffs. In this way, irrigation fluid can be introduced through the connector 41 and to the isolated region between the two cuffs, and the introduced fluid (together with the dispersed secretions) can be aspirated through the port 46 and ultimately out through the connector 43.

In some cases, it will be desirable to minimize the volume of the isolated region formed between the primary occlusion cuff and the secondary isolation cuff. That can be achieved most simply by moving the two cuffs more closely together. The ability to move the balloons together is limited somewhat when either or both of the cuffs have shoulders 21 or 23 projecting into the space therebetween. One way of significantly reducing the volume is to use everted cuff configurations, as shown in FIG. 1A. The ventilator tube 12 can have an isolation cuff 20a with an everted end 25 facing an everted end 27 of the isolation cuff 22a. The everted ends are sealed against the ventilation tube 12 as shown in the shaded regions, and the aspiration port 46a is disposed in the very small volume remaining between the balloons. Creation of a small volume can be advantageous since it both reduces the volume of irrigation fluid that is needed and enhances removal efficiency (since it is easier to thoroughly was the smaller volume).

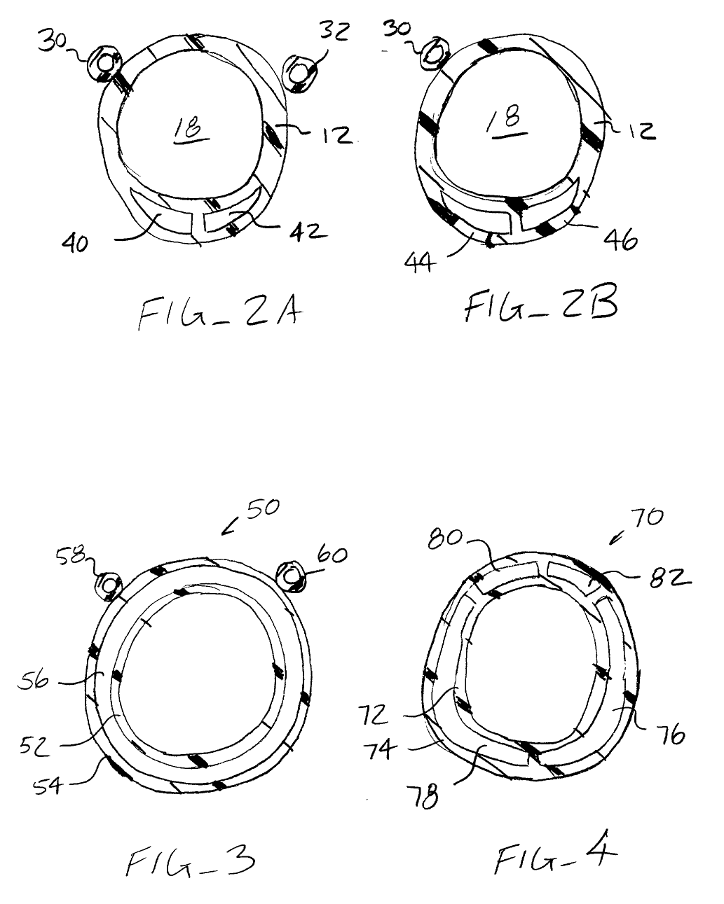

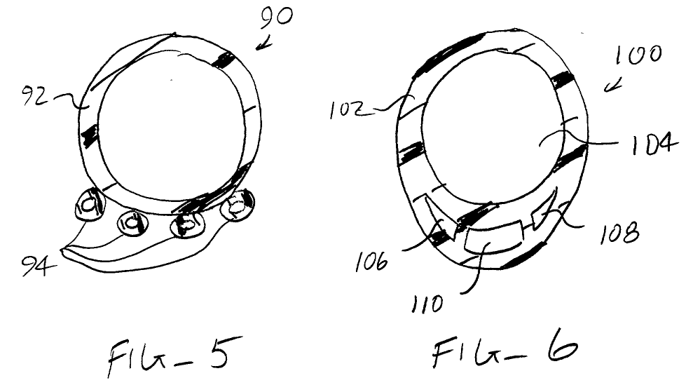

Referring now to FIG. 3, the ventilation devices of the present invention may optionally include a single irrigation/aspiration lumen. As illustrated in FIG. 3, a device 50 comprises coaxially tubes 52 and 54 which together define an annular irrigation/aspiration lumen 56. Tubes 58 and 60 may be provided externally for inflation of the primary occlusion cuff and secondary isolation cuff (not shown).

As a further alternative, a ventilation device 70 may comprise coaxially tubes 72 and 74 having an annular region therebetween which is divided into separate lumens, such as irrigation lumen 76, aspiration lumen 78, first inflation lumen 80, and second inflation lumen 82.

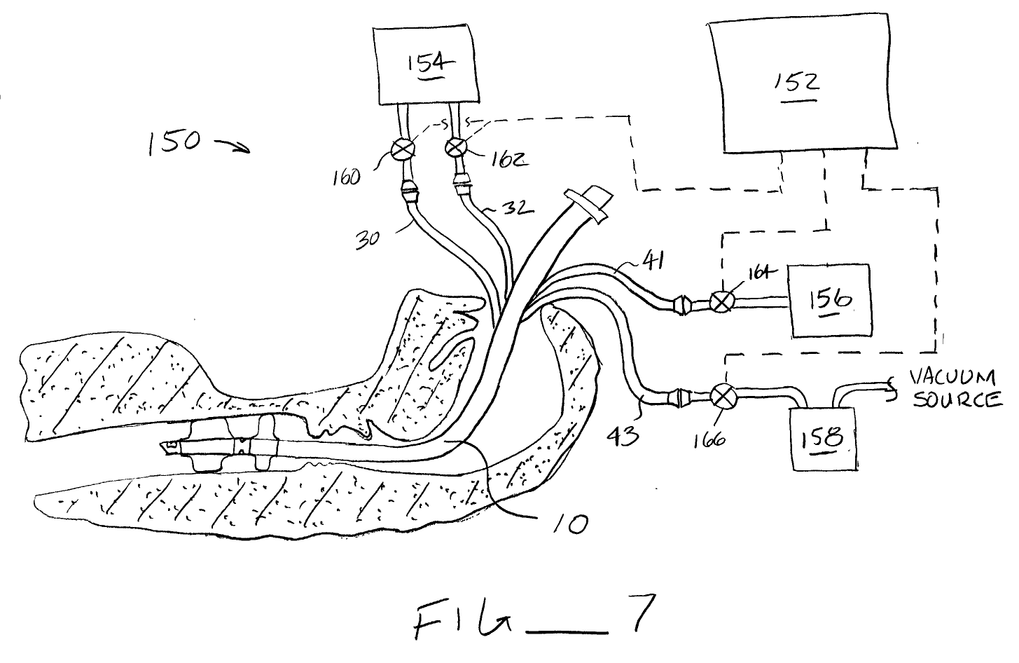

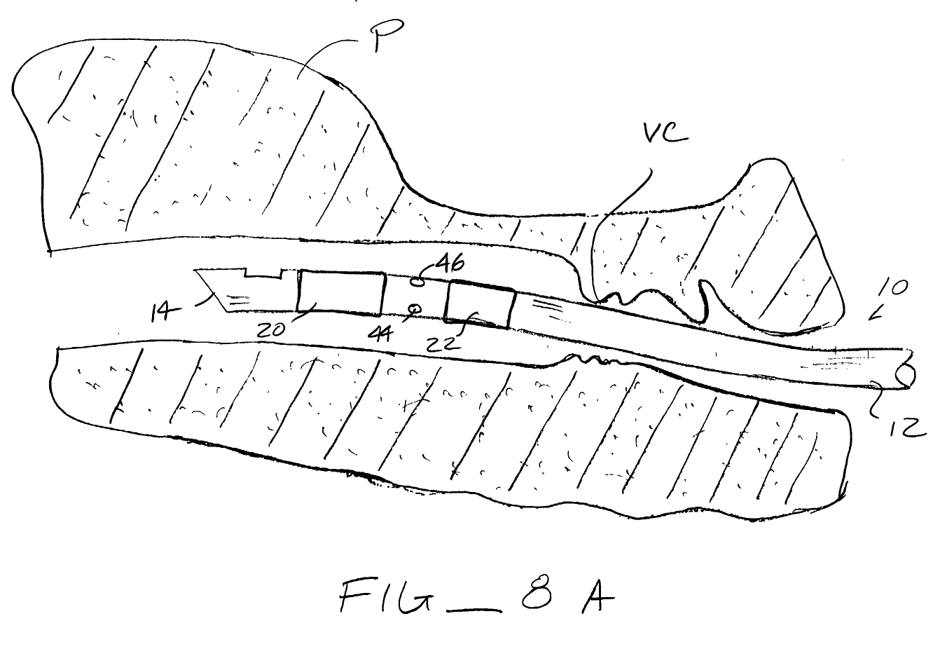

A still further alternative embodiment is shown in FIG. 5. A ventilation device 90 comprises a ventilation tube 92 having no lumens formed in its wall. Instead each of the irrigation, aspiration, and ventilation lumen is formed as a separate external tube 94 on the outside of the device.

As a still further alternative, a ventilation device 100 may comprise a ventilation tube 102 consisting of a single extrusion having a primary ventilation lumen 104, a first cuff inflation lumen 106, a second cuff inflation lumen 108, and a single irrigation/aspiration lumen 110. The oval profile of both device 10 and 100 is advantageous since it conforms to the space available as the ventilation tube passes through the vocal cords in the trachea, as illustrated hereinafter.

Thus far, the ventilation devices have been illustrated as endotracheal tubes, i.e. devices which are intended to pass through the patient's mouth or nasal passages and into the throat and trachea. The ventilation devices of the present invention are also intended to include tracheostomy tubes which are introduced through a small transcutaneous incision in the trachea and are used primarily for long term placement and ventilation.

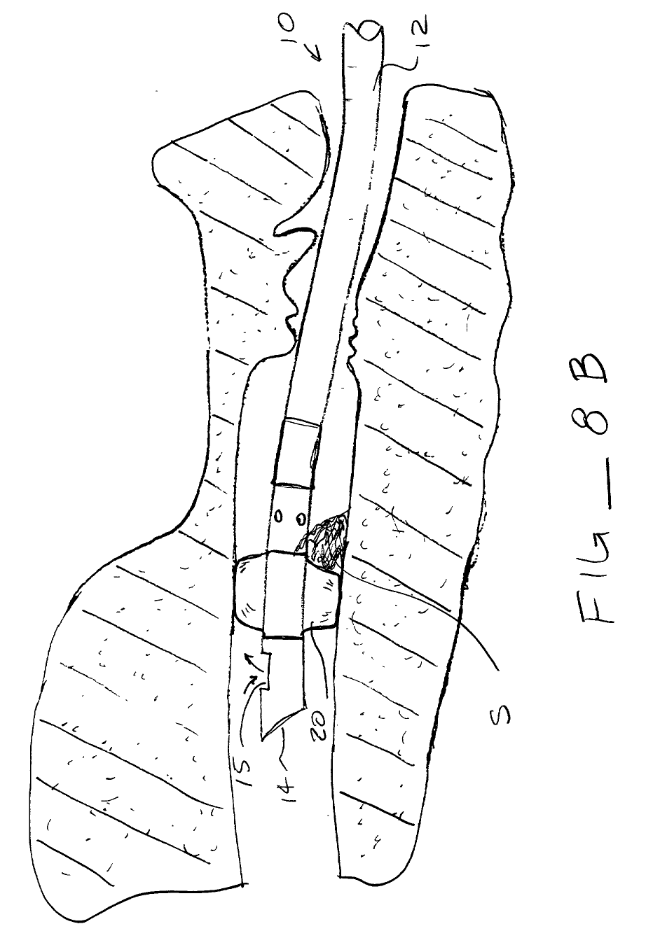

Referring now to FIG. 7, a system 150 according to the present invention comprises a ventilation device, such as ventilation device 10, in combination with a controller 152, an inflation medium source 154, an irrigation medium source 156, and a vacuum disposal source 158. The controller may be any type of digital, electromechanical, or other control system which permits the real time monitoring and control of the valves, pressures, flow rates, and other aspects of the ventilation system of the present invention. The inflation source 154 may be a pressure regulator (when used in combination with a high pressure gas source, such as pressurized gas sources commonly available in hospitals and other treatment facilities), or may further comprise of selfcontained pump and regulator assembly in order to maintain a constant source of pressurized inflation medium. The pressurized medium source 154 will be connected to the inflation connectors 30 and 32 of ventilation device 10, typically through controllable on/off valves, such as solenoid valves 160 and 162. The valves will typically also include means for relieving pressure from the associated inflation cuffs when the inflation medium is turned off. Optionally, pressure and control means may be provided to monitor pressure and assure that the balloons are not subject to excessive pressures.

The source of irrigation medium 156 will be connected to the irrigation lumen connector 41, typically through another on/off valve 164. The vacuum disposal source 158 will be connected to the aspiration connector 43, also through and on/off valve 166. Controller 152 will be connected to each of the valves 160-166 in order to open and close the valves in accordance with a desired program, as discussed in more detail below. Optionally, the controller will also include timers, pressure control means, pressure monitoring means, flow control and/or monitoring means, and other capabilities useful in monitoring performance of the ventilation system and the associated secretion washing systems.

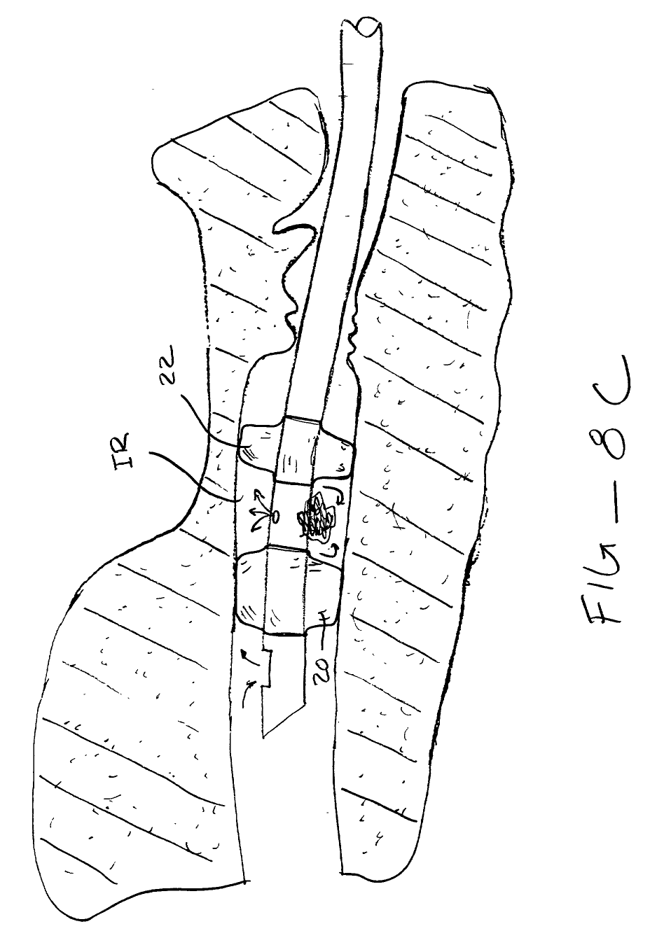

Referring now to FIGS. 8A-8C, ventilation device 10 is first introduced to the patient P with both cuffs 20 and 22 deflated. The cuffs 20 and 22 are positioned past the vocal cords (VC), as illustrated in FIG. 8A. The primary occlusion cuff 20 will then be inflated, as illustrated in FIG. 8B, in order to isolate the lower regions of the respiratory system to facilitate mechanical ventilation. Air will then be introduced to the patient's lungs through the ventilation port 15 located near the distal end 14 of the device 10. Control of the inflation of occlusion cuff 20 will usually, but not necessarily, be controlled by the control system of the present invention. As described thus far, deployment of the ventilation device is generally conventional, and secretions will accumulate on the proximal side of the occlusion balloon 20, as generally shown at S in FIG. 8B.

In order to remove the secretions S according to the method at present invention, the secondary isolation balloon 22 will be inflated to create an isolated region IR between cuff 22 and cuff 20, as illustrated in FIG. 8C. Patient ventilation will be maintained at all times during this cleaning process which is about to be described. After the secondary isolation cuff 22 is inflated, an irrigation solution will be introduced through the port 44. Typically, the irrigation solution will be introduced at a rate from about 1 cc/minute to 200 cc/minute to a total volume to 3 cc to 10 cc. Aspiration will be initiated in the isolated region IR either after the irrigation solution has been introduced or simultaneously with introduction of the irrigation solution. In either case, the irrigation solution will tend to wash, disperse, and in some instances solubilize the materials in the accumulated secretions S to facilitate their collection as the irrigation solution is aspirated through the port 46.

While it is preferred to use an irrigation solution to facilitate washing of the secretions, the present invention will find use with aspiration alone performed in the isolation region IR. It will be appreciated that isolation cuff 22 can still be used to perform the isolated region IR, and in some instances it will be sufficient then to aspirate using a port, or optionally a tube extending from the port (not illustrated) in order to remove the secretions. Generally, however, it will be much preferred to use an irrigation solution in combination with aspiration.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A ventilation device comprising:
a ventilation tube having a proximal end and a distal end;
an inflation cuff near the distal end of the ventilation tube; and
an isolator barrier on the ventilation tube spaced proximally of the inflatable cuff;
wherein the ventilation tube has an aspiration lumen with an external port near the proximal end of the ventilation tube and an internal port between the inflatable cuff and the isolator barrier.

2. A ventilation device as in claim 1, wherein the aspiration lumen has two external ports which may be separately connected to an aspiration source and an irrigation source.

3. A ventilation device as in claim 1, wherein the aspiration lumen comprises a separate tube.

4. A ventilation device as in claim 1, wherein the ventilation tube has an irrigation lumen with an external port near the proximal end of the ventilation tube and an internal port between the inflatable cuff and the isolation barrier.

5. A ventilation device as in claim 4, wherein the irrigation lumen comprises a separate tube.

6. A ventilation device as in claim 1, wherein the isolation barrier comprises a second inflatable cuff.

7. A ventilation device as in claim 6, wherein the first and second inflatable cuffs are axially spaced apart by a distance in the range from 0.25 cm to 10 cm.

8. A system comprising:
(a) a ventilation device as in claim 6;
(b) a source of inflation medium;
(c) a source of irrigant solution;
(d) a vacuum disposal source; and
(e) a controller which selectively inflates the secondary isolation barrier inflatable cuff, delivers irrigation solution to an isolated region between the inflatable cuff and the isolation barrier, and aspirates the irrigant to the vacuum disposal source.

9. A system as in claim 8, wherein the controller controls delivery of the irrigation solution and aspiration of the irrigation solution sequentially.

10. A system as in claim 8, wherein the controller delivers the irrigation solution and aspirates the irrigation solution simultaneously.

11. A system as in claim 8, wherein the controller operates on a duty cycle of from 10 seconds to 10 minutes.

12. A method for removing accumulated secretions from over a ventilation tube cuff present in a patient's trachea, said method comprising:
(a) deploying an isolation barrier above the cuff to isolate a region in the trachea between the barrier and the cuff; and
(b) aspirating accumulations captured in the isolated region.

13. A method as in claim 12, comprising repeating step (b) periodically while the isolation barrier remains deployed.

14. A method as in claim 12, further comprising:
(c) opening the isolation barrier to permit further accumulations to collect over the cuff.

15. A method as in claim 14, comprising repeating steps (a) through (c) periodically.

16. A method as in claim 15, wherein steps (a) through (c) are repeated at intervals in the range from 10 seconds to 10 minutes.

17. A method as in claim 12, wherein deploying an isolation barrier comprises inflating a second inflatable cuff which is spaced-apart from the endotracheal tube cuff.

18. A method as in claim 12, further comprising irrigating the isolated region above the cuff.

19. A method as in claim 18, wherein the isolated region is irrigated while it is being aspirated.

20. A method as in claim 18, wherein the isolated region is irrigated prior to aspirating.

21. An improved method for removing accumulations from over an endotracheal tube cuff in a patient's trachea, said method being of the type wherein the accumulations are aspirated from above the cuff, wherein the improvement comprises:
isolating a region above the cuff prior to aspirating the accumulations.

22. An improved method as in claim 21, wherein the improvement further comprises:
introducing a washing fluid over the cuff prior to aspirating, wherein the fluid and accumulations are aspirated together.

23. A kit comprising:
a ventilation tube having an inflatable cuff and an isolation barrier; and
instructions for use setting forth the method of claim 12.

24. A kit as in claim 23, further comprising a package holding both the ventilation tube and the instructions for use.
